(19) United States
(12) Patent Application Publication
(10) Pub. No.: US 2008/0300461 A1
(43) Pub. Date: Dec. 4, 2008

(54) Endoscopic Device

(75) Inventors: William D. Shaw, Cincinnati, OH (US); Jeffrey D. Messerly, Cincinnati, OH (US); Gary W. Knight, West Chester, OH (US); Jessica Fox, Mt. Vernon, IN (US)

Correspondence Address: WELSH & FLAXMAN LLC, 2000 DUKE STREET, SUITE 100, ALEXANDRIA, VA, 22314, US

(73) Assignee: Ethicon Endo-Surgery, Inc., Cincinnati, OH (US)

(21) Appl. No.: 11/755,926

(22) Filed: May 31, 2007

(51) Int. Cl. A61B 1/008

(52) U.S. Cl. 600/141

(57) ABSTRACT

An endoscopic surgical instrument for performing a procedure inside a patient. The instrument includes a handle assembly including a grip for holding the instrument and controls for controlling movement of the instrument. An elongate shaft having a grip end is mounted on the handle assembly. The shaft has a working end opposite the grip end. The shaft has a proximal portion adjacent the grip end and a distal portion adjacent the working end. The proximal portion has a centerline and is selectively rotatable with respect to the handle assembly. The distal portion has a centerline and is selectively articulatable relative to the proximal portion of the shaft. The instrument includes an end effector mounted on the working end of the elongate shaft. The end effector is selectively rotatable about the distal portion of the shaft.

### BACKGROUND

The present invention relates generally to an endoscopic device, and more particularly too an endoscopic device capable of independent articulation, rotation and actuation.

Endoscopic devices are used for examining and performing surgery inside a patient's body. Endoscopic devices typically include an elongate shaft for entering the body through a natural orifice or an incision and for extending into and through a body canal, an internal cavity or an organ so surgery can be performed on the interior of the body without making large incisions in the patient. Endoscopic devices permit less invasive surgeries than conventional examination and surgical methods. The endoscopic devices also typically have some end effector for performing a specific task. For example, some end effectors apply fasteners to tissue. Other end effectors are used to retract tissue. Still other end effectors include forceps for grasping and manipulating tissue and devices used by the physician.

It is desirable for endoscopic devices to be highly manipulatable so that the end effector can be easily positioned at precise locations in the patient. For this reason, it is desirable for the endoscopic devices to have multiple degrees of freedom (i.e., be adapted to move in several directions). However, it is not uncommon for devices having multiple degrees of freedom to be complicated to manufacture and use. Such devices can also be bulky, which is an undesirable characteristic for endoscopic devices. Thus, there is a need for an endoscopic device that can be easily manipulated in several directions while maintaining a small size to allow endoscopic delivery to sites within the patient.

### BRIEF SUMMARY

The present invention relates an endoscopic surgical instrument for performing a surgical procedure inside a patient. The instrument comprises a handle assembly including a grip for holding the instrument and a plurality of controls for controlling movement of the instrument. Further, the instrument includes an elongate shaft having a grip end mounted on the handle assembly and a working end opposite the grip end. The shaft has a proximal portion adjacent the grip end and a distal portion adjacent the working end. The proximal portion has a centerline and is selectively rotatable with respect to the handle assembly about the centerline of the proximal portion of the shaft. The distal portion has a centerline and is selectively articulatable relative to the centerline of the proximal portion of the shaft. In addition, the instrument comprises an end effector mounted on the working end of the elongate shaft. The end effector is selectively rotatable about the centerline of the distal portion of the shaft.

In another aspect, the present invention includes an endoscopic surgical instrument for performing a surgical procedure inside a patient. The instrument comprises a handle assembly including a grip for holding the instrument and a plurality of controls for controlling operation and movement of the instrument. Further, the instrument comprises an elongate shaft having a grip end mounted on the handle assembly and a working end opposite the grip end. The shaft has a proximal portion adjacent the grip end that is selectively moveable with respect the handle assembly and a distal portion adjacent the working end that is selectively moveable with respect to the proximal end. Moreover, the instrument includes an end effector mounted on the working end of the elongate shaft. The end effector is selectively operable and selectively moveable with respect to the distal portion of the shaft.

Other aspects of the present invention will be in part apparent and in part pointed out hereinafter.

### BRIEF DESCRIPTION OF THE DRAWINGS

FIG. 1 is a schematic side elevation of a endoscopic instrument of the present invention;

FIG. 2 is a schematic detail in partial section showing interior features of the endoscopic instrument;

FIG. 3 is a schematic cross section of a portion of the endoscopic instrument;

FIG. 4 is a schematic cross section of another portion of the endoscopic instrument;

FIG. 5 is a schematic cross section of yet another portion of the endoscopic instrument; and

FIG. 6 is a schematic detail of another portion of the endoscopic instrument.

Corresponding reference characters indicate corresponding parts throughout the several views of the drawings.

### DETAILED DESCRIPTION

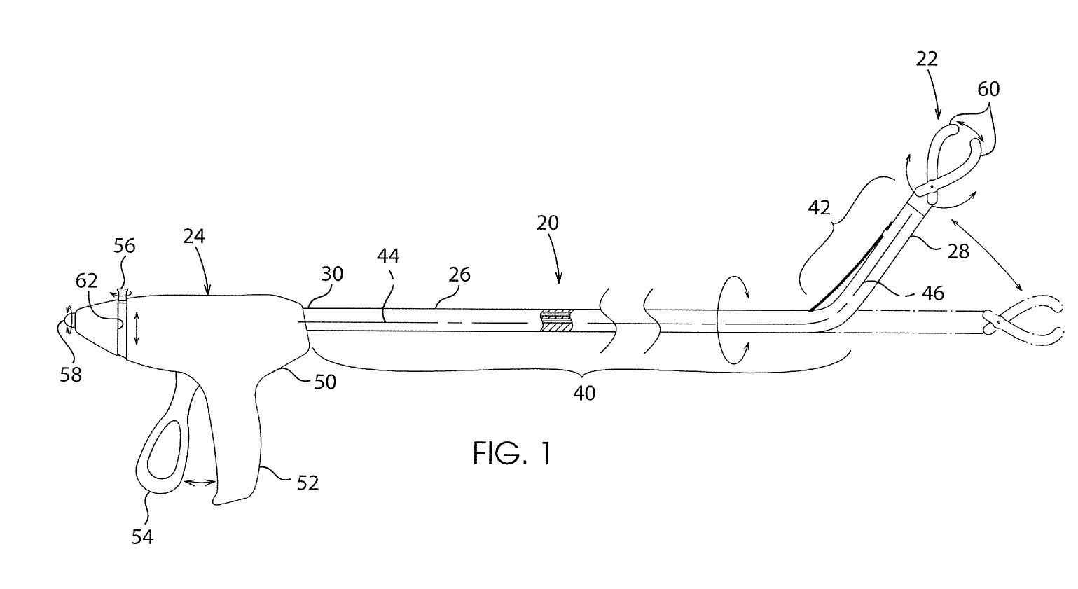

Referring now to the drawings and in particular FIG. 1, an endoscopic surgical instrument of the present invention is generally designated by the reference number 20. The instrument 20 is specifically adapted for performing endoscopic surgery. The instrument 20 generally includes an end effector (generally designated by 22) and a handle assembly (generally designated by 24) mounted on opposite ends of an elongate shaft 26. The shaft 26 has a working end 28, on which the end effector 22 is mounted, and a grip end 30, on which the handle assembly 24 is mounted.

In one embodiment, the shaft 26 is a hollow tube having a circular cross section, a length of between about 30 cm and about 225 cm, and an outside diameter of between about 2.8 mm and about 10 mm. Although the shaft 26 may be made of other materials without departing from the scope of the present invention, in one embodiment the shaft is made of stainless steel flex coil. The shaft 26 includes a proximal portion 40 adjacent the grip end 30 and a distal portion 42 adjacent the working end 28. Further, the proximal portion 40 has an imaginary centerline 44 extending longitudinally along its length and the distal portion 42 has an imaginary centerline 46 extending along its length. As will be explained in more detail below, the distal portion 42 is selectively articulatable with respect to the proximal portion 40 of the shaft 26 so the centerline 46 of the distal portion is angled with respect to the centerline 44 of the proximal portion. Further, the proximal portion 40 of the shaft 26 is selectively rotatable about its centerline 44 with respect to the handle assembly 24.

As further illustrated in FIG. 1, the handle assembly 24 includes a housing 50 having a grip 52 for holding the instrument and several controls 54, 56, 58 for controlling movement of the instrument 20. The first control 54 is a lever pivotally attached to the housing 50. The lever 54 is pivotable to operate the end effector 22. For example, in the case of a grasping end effector 22 having opposing jaws 60 such as shown in FIG. 1, the jaws are opened and closed by moving the lever 54 rearward and forward, respectively. The second control 56 is a knob mounted in a slot 62 in the housing 50 for rotation and sliding movement along the slot. The second control knob 56 rotates about its axis to articulate the distal portion 42 of the shaft 26 between a straight orientation as shown in phantom in FIG. 1 and an angled orientation as shown in solid lines in FIG. 1. Further, the knob 56 may be moved along the slot 62 to rotate the proximal portion 40 of the shaft 26 about its centerline 44 relative to the handle assembly 24. The third control 58 is a knob mounted on the housing 50 for rotation. As the third control knob 58 is rotated, the end effector 22 rotates about the centerline 46 of the distal portion 42 of the shaft 26.

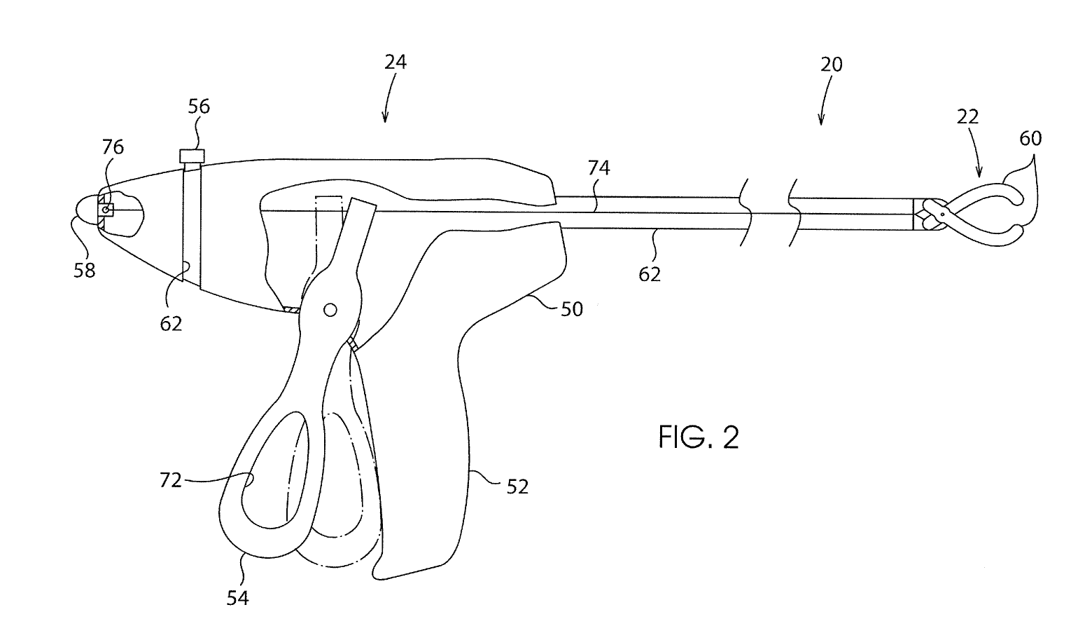

As shown in FIG. 2, the handle assembly 24 includes the molded housing 50. Although the housing 50 may be made of other materials without departing from the scope of the present invention, in one embodiment the housing is made of polycarbonate. As further shown in FIG. 2, the first control lever 54 is pivotally mounted in the housing 50 on a pin 70. Further, the lever 54 may include an opening 72 for receiving a digit (e.g., a thumb) of the surgeon. The lever 54 engages a control wire 74 that extends from the third control knob 58 to the end effector 22. The knob 58 is rotatably mounted on the housing 50 and may include a bushing or bearing (not shown) to reduce friction between the knob and housing. One end of the control wire 74 is connected to the knob 58. Although other connection means may be used without departing from the scope of the present invention, in one embodiment, the wire 74 is connected to the knob 58 with a screw fastener 76. The end of the control wire 74 opposite the knob 58 is connected to the end effector 22.

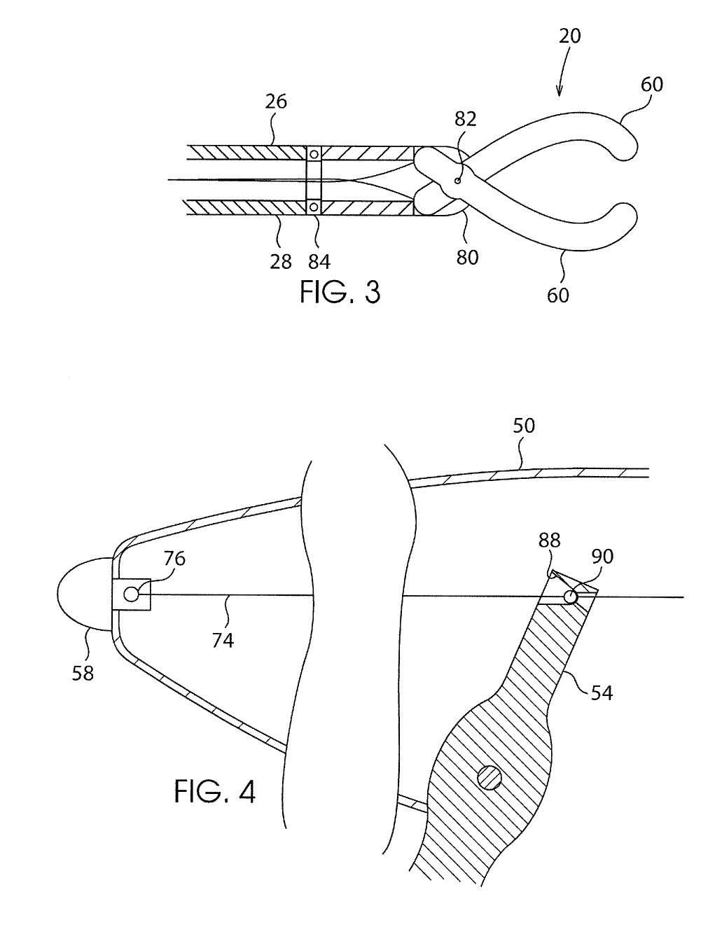

As illustrated in FIG. 3, the end effector 22 of one embodiment includes a pair of opposing jaws 60 connected to a clevis 80 by a pin 82. The clevis 80 is mounted on a bearing 84 that is mounted on the distal portion 42 of the shaft 26. The control wire 74 splits in half near the end effector 22. Each half of the control wire 74 is connected to one of the opposing jaws 60 so that as the wire is pulled the jaws close, and as the wire is pushed the jaws open. Further, as will be apparent to those skilled in the art, as the control knob 58 is rotated, the control wire 74 rotates, turning the wires and the end effector 22 about the centerline 46 of the distal portion 42 of the shaft 26. Although the end effector 22 may be made of other materials without departing from the scope of the present invention, in one embodiment the end effector is made from stainless steel.

FIG. 4 shows a detail of the control lever 54. The control lever 54 includes a yoke 90 that engages a bead or knot 92 mounted on the control wire 74. As will be understood by those skilled in the art, the lever 54 pulls the wire 74 to close the jaws 60 of the end effector 22 when an upper end of the lever moves toward the control knob 58, and the lever pushes the wire to open the jaws of the end effector when the upper end of the lever moves away from the control knob 58. In one embodiment, the jaws 60 of the end effector 22 are biased toward the open position so they act to move away from the control knob 58.

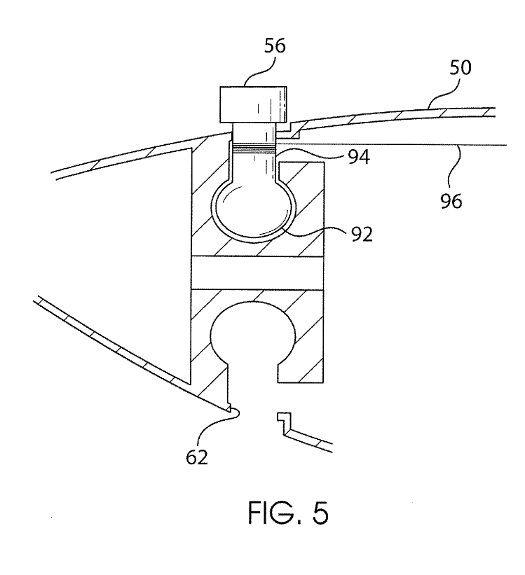

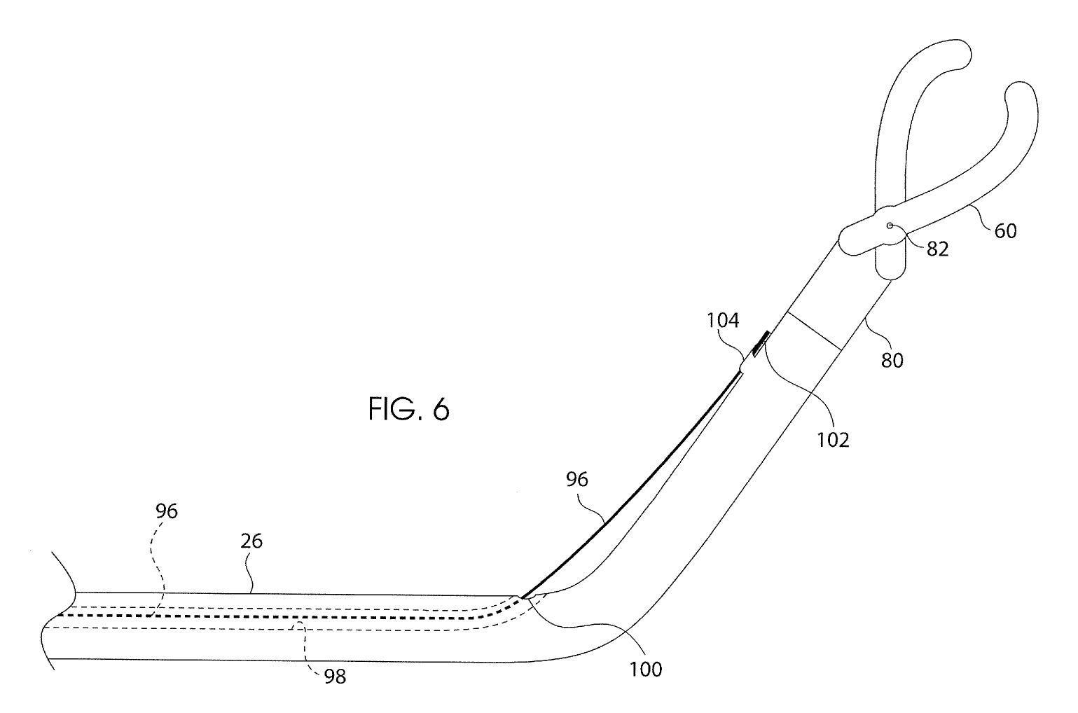

FIG. 5 illustrates a partial cross section of the handle assembly housing 50. The housing 50 includes a slot 90 that receives a spherical end 92 of the control knob 56. The knob 56 includes a spool 94 on which a control cable 96 is wound. As shown in FIG. 6, the control cable 96 passes through a laterally offset passage 98 in the proximal portion 40 of the shaft 26. The passage 98 ends at an opening 100 in the shaft 26 and the control cable 96 extends along the outside of the shaft to a termination point 102 on the distal portion 42 of the shaft. Although the termination point 102 may be formed in other ways without departing from the scope of the present invention, in one embodiment the cable 96 is attached to the shaft 26 by a band 104 and adhesive (not shown). As will be apparent to those skilled in the art, as the knob 56 is turned, the cable 96 winds or unwinds on the spool 94. As the cable 96 is wound on the spool 94, the distal portion 42 of the shaft 26 articulates relative to the proximal portion 40 of the shaft. Further, as the knob 56 is moved along the slot 62, the cable 96 rotates the proximal portion 40 of the shaft 26 so it rotates about its centerline 44. It is envisioned that the proximal portion 40 of the shaft 26 may be mounted on the housing 50 with bearings (not shown) to improve the ease with which the proximal portion rotates about its centerline 44.

As will be apparent to those skilled in the art, the lever control 54 forms an end effector operation control. Further, the knob control 56 forms both a proximal shaft portion 44 rotation (or motion) control and a distal shaft portion 42 articulation (or motion) control. The knob control 58 forms an end effector rotation (or motion) control.

Based on the description above, the operation of the instrument 20 will be apparent to those skilled in the art. The endoscopic instrument 20 described above may be made so that the end effector 22 and shaft 26 are not bulky, thereby improving potential outcome and reducing a potential for trauma. Further, the construction is simple, reducing initial manufacturing cost.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. An endoscopic surgical instrument for performing a surgical procedure inside a patient, said instrument comprising:
a handle assembly including a grip for holding the instrument and a plurality of controls for controlling movement of the instrument;
an elongate shaft having a grip end mounted on the handle assembly and a working end opposite said grip end, said shaft having a proximal portion adjacent said grip end and a distal portion adjacent said working end, said proximal portion having a centerline and being selectively rotatable with respect to the handle assembly about the centerline of the proximal portion of the shaft, said distal portion having a centerline and being selectively articulatable relative to the centerline of the proximal portion of the shaft; and
an end effector mounted on the working end of the elongate shaft, said end effector being selectively rotatable about the centerline of the distal portion of the shaft.

2. An instrument as set forth in claim 1 wherein at least one control of said plurality of controls comprises an end effector rotation control operatively connected to the end effector for rotating the end effector about the centerline of the distal portion of the shaft.

3. An instrument as set forth in claim 2 further comprising a wire extending from the end effector to one control of said plurality of controls on the handle assembly, said control being operable to rotate the wire thereby rotating the end effector about the centerline of the distal portion of the shaft.

4. An instrument as set forth in claim 3 further comprising a bearing mounted between the proximal portion of the shaft and the end effector.

5. An instrument as set forth in claim 1 wherein at least one control of said plurality of controls comprises a shaft articulation control operatively connected to the distal portion of the shaft for articulating the distal portion of the shaft relative to the centerline of the proximal portion of the shaft.

6. An instrument as set forth in claim 5 further comprising a wire extending from the distal portion of the shaft to one control of said plurality of controls on the handle assembly, said control being operable to tension the wire thereby articulating the distal portion of the shaft relative to the centerline of the proximal portion of the shaft.

7. An instrument as set forth in claim 1 wherein at least one control of said plurality of controls comprises a proximal portion rotation control operatively connected to the proximal portion of the shaft for rotating the proximal portion of the shaft relative to the handle assembly about the centerline of the proximal portion of the shaft.

8. An instrument as set forth in claim 7 further comprising a wire extending from the proximal portion of the shaft to one control of said plurality of controls on the handle assembly, said control being operable to rotate the wire thereby rotating the proximal portion of the shaft relative to the handle assembly.

9. An endoscopic surgical instrument for performing a surgical procedure inside a patient, said instrument comprising:
a handle assembly including a grip for holding the instrument and a plurality of controls for controlling operation and movement of the instrument;
an elongate shaft having a grip end mounted on the handle assembly and a working end opposite said grip end, said shaft having a proximal portion adjacent said grip end that is selectively moveable with respect the handle assembly and a distal portion adjacent said working end that is selectively moveable with respect to the proximal end; and
an end effector mounted on the working end of the elongate shaft, said end effector being selectively operable and selectively moveable with respect to the distal portion of the shaft.

10. An instrument as set forth in claim 9 wherein at least one control of said plurality of controls comprises an end effector operation control operatively connected to the end effector for operating the end effector.

11. A surgical instrument as set forth in claim 9 wherein:
the handle assembly comprises a housing;
the control comprises a lever pivotally attached to the housing; and
the instrument further comprises a mechanism operatively connecting the lever to said end effector to selective operate the end effector in response to pivoting the lever.

12. An instrument as set forth in claim 9 wherein at least one control of said plurality of controls comprises an end effector motion control operatively connected to the end effector for moving the end effector with respect to the distal portion of the shaft.

13. An instrument as set forth in claim 12 wherein the control comprises an end effector rotation control operatively connected to the end effector for rotating the end effector about the centerline of the distal portion of the shaft.

14. An instrument as set forth in claim 13 wherein the end effector rotation control comprises a knob rotatably mounted on the handle assembly.

15. An instrument as set forth in claim 9 wherein at least one control of said plurality of controls comprises a distal shaft portion motion control operatively connected to the distal portion of the shaft for moving the distal portion with respect to the proximal portion of the shaft.

16. An instrument as set forth in claim 15 wherein the control comprises a distal shaft portion articulation control operatively connected to the distal portion of the shaft for articulating the distal portion with respect to the proximal portion of the shaft.

17. An instrument as set forth in claim 16 wherein the distal shaft portion articulation control comprises a knob rotatably mounted on the handle assembly.

18. An instrument as set forth in claim 9 wherein at least one control of said plurality of controls comprises a proximal shaft portion motion control operatively connected to the proximal portion of the shaft for moving the proximal portion with respect to the handle assembly.

19. An instrument as set forth in claim 18 wherein the control comprises a proximal shaft portion rotation control operatively connected to the proximal portion of the shaft for rotation with respect to the handle assembly.

20. An instrument as set forth in claim 16 wherein the proximal shaft portion rotation control comprises a knob slidably mounted on the handle assembly.
